(19) United States
(12) Patent Application Publication
(10) Pub. No.: US 2025/0121189 A1
(43) Pub. Date: Apr. 17, 2025

(54) VENTRAL SPINAL CORD STIMULATION FOR AT LEAST PARTIAL RESTORATION OF MOTOR FUNCTION

(71) Applicant: Case Western Reserve University, Cleveland, OH (US)

(72) Inventors: Gustaf M. Van Acker, Shaker Heights, OH (US); Chong Kim, Bay Village, OH (US); Chris Pulliam, Cleveland, OH (US); Nathan Makowski, Cleveland, OH (US)

(21) Appl. No.: 18/914,696

(22) Filed: Oct. 14, 2024

Related U.S. Application Data
(60) Provisional application No. 63/590,039, filed on Oct. 13, 2023.

(51) Int. Cl. A61N 1/36; A61N 1/02; A61N 1/05

(52) U.S. Cl. CPC A61N 1/36062; A61N 1/025; A61N 1/0551; A61N 1/36003; A61N 1/36132; A61N 1/36139

(57) ABSTRACT

A motor function of a subject can be at least partially restored via electrical stimulation. One or more stimulating electrodes can be positioned within a ventral space of a spinal column of the subject to deliver the electrical stimulation to a motor fiber in the spinal column to at least partially restore the motor function. The one or more stimulating electrodes can be in electrical communication with a signal generator. A controller can be connected to the signal generator, and optionally, one or more sensing elements. The controller can receive a signal comprising at least one input, which can be manual or from the one or more sensing elements; configure the electrical signal to stimulate motor fiber(s) in a descending tract of the spinal cord of the subject based on the at least one input; and send the electrical signal to the signal generator to generate the electrical stimulation.

### CROSS-REFERENCE TO RELATED APPLICATIONS

This application claims the benefit of U.S. Provisional Application No. 63/590,039, entitled “VENTRAL SPINAL CORD STIMULATION FOR AT LEAST PARTIAL RESTORATION OF MOTOR FUNCTION,” filed 13 Oct. 2023, which is hereby incorporated by reference in its entirety for all purposes.

### TECHNICAL FIELD

The present disclosure relates to restoring motor function, and more specifically, to systems and methods for at least partially restoring motor function in patients suffering from stroke, spinal cord injury (SCI), and/or another disorder of the central nervous system (CNS), via ventral spinal cord stimulation (SCS).

### BACKGROUND

Impairment of one or more motor functions can have a drastic negative affect on a patient's quality of life. Motor functions are controlled by contraction of muscles (voluntary and/or involuntary) due to neural impulses. The neural impulses are generated in the brain and transmitted from the brain through the spinal cord to one or more motor neurons. In response to the neural impulses the one or more motor neurons release neurotransmitters at neuromuscular junctions and the muscles can contract in response to the neurotransmitters triggering a chemical reaction within the muscle cells. Various conditions (such as stroke and spinal cord injury) can at least partially block the conduction of the neural impulses between the brain and the muscle, impairing one or more motor functions. Traditionally, restoring the impaired one or more motor functions after a stroke, spinal cord injury, and/or another disorder of the central nervous system (CNS), can be difficult and, in severe cases, impossible.

### SUMMARY

Motor function can be at least partially restored after stroke, spinal cord injury, and/or another disorder of the central nervous system (CNS), by ventral spinal cord stimulation (SCS). Ventral SCS can deliver an electrical stimulation via at least one electrode within the ventral space of the spinal column (e.g., one or more electrodes at one or more levels or sides of the spinal column). The electrical stimulation can be configured and/or delivered based on a manual input and/or one or more signals from one or more sensors that can detect an intent to perform a motor function.

A system is described to at least partially restore motor function in a patient suffering from a stroke, spinal cord injury, and/or other conditions of the CNS. One or more sensing elements can be configured to record one or more physiological signals from a subject. A signal generator can be configured to produce at least one electrical signal. One or more stimulating electrodes can be configured to be positioned within a ventral space of a spinal column of the subject and in electrical communication with the signal generator. A controller can be connected to the one or more sensing elements and to the signal generator and can comprise: a non-transitory memory configured to store instructions, and a processor configured to execute the instructions to at least: receive a signal comprising at least one recorded physiological signal from at least one of the one or more sensing elements; configure at least one electrical signal to stimulate at least one motor fiber in a descending tract of the spinal cord of the subject based on the at least one recorded physiological signal; and send the at least one electrical signal to the signal generator to be applied through at least one of the one or more stimulating electrodes. The at least one of the one or more stimulating electrodes are configured to apply the at least one electrical signal to the at least one motor fiber to at least partially restore a motor function of the subject.

A method is described for at least partially restoring motor function in the patient suffering from a stroke, spinal cord injury, and/or other conditions of the CNS. Steps of the method can be performed by a controller comprising a processor and can include receiving an input based on at least one motor function of the subject; configuring at least one electrical signal designed to stimulate at least one motor fiber in a descending tract of the spinal column and/or spinal cord; and sending a configuration of the at least one electrical signal to a signal generator. The at least one electrical signal is transmitted from the signal generator to at least one stimulating electrode within a ventral space of a spinal column of a subject affected by stroke and/or spinal cord injury for application to the at least one motor fiber to at least partially restore the at least one motor function of the subject.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing a system that can be used to provide ventral spinal cord stimulation (SCS);

FIG. 2 shows example placements of one or more stimulating electrodes of the system of FIG. 1 with respect to the spinal cord;

FIG. 3 is a diagram showing another system that can be used for both dorsal and ventral SCS;

FIG. 4 shows an example process flow diagram of a method for restoring at least a portion of motor function with ventral SCS;

FIG. 5 shows an example process flow diagram of a method for combining ventral and dorsal SCS; and

FIG. 6 shows an example process flow diagram of a method for controlling ventral SCS with feedback.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

As used herein, the term “motor function” refers to any activity (glandular activity, involuntary muscular activity, voluntary muscular activity) that is caused by a stimulation (e.g., biological electrical signals from one or more motor neurons or the like and/or extrinsic stimulation).

As used herein, the term “extrinsic” when used with stimulation can refer to a non-biologically created stimulation. One example of extrinsic stimulation is electrical stimulation, by which an electrical signal is delivered to a neural structure by an electrode. Other non-limiting examples of extrinsic stimulation can include pharmacological stimulation, light stimulation, heat stimulation, mechanical stimulation, or the like that are applied by an artificial stimulating device.

As used herein, the term “motor fiber” refers to a neural fiber that can conduct a neural impulse and/or electrical signal to one or more muscles to cause a reaction (e.g., contraction) in the one or more muscles that results in a motor function.

As used herein, the term “spinal cord stimulation”, also referred to as “SCS”, refers to a type of electrical stimulation in which an electrical signal is specially configured (e.g., by a controller), generated (e.g., by a signal generator), and delivered to one or more neural structures in the spinal cord by one or more electrodes. Each of the one of more electrodes can be positioned in epidural space adjacent the spinal cord to apply the electrical signal to one or more neural fibers in the spinal cord that are part of a neural pathway to restore motor function. SCS may be dorsal SCS and/or ventral SCS depending on the spinal pathway being targeted. In some instances, the SCS can be due to and/or an intensity of the SCS can be determined (e.g., by a controller) based on one or more signals from one or more sensors received by the controller.

As used herein, the term “electrical signal” refers to a transmission that conveys information about an electric phenomenon (e.g., current, voltage, etc.), which varies with time and/or space, to one or more neural fibers. The electrical signal can be, for example, an alternating current signal that varies in amplitude for at least a portion of time and/or a direct current (e.g., constant amplitude for at least a portion of time) signal. It is believed that there are lower activation thresholds to activate motor fibers than sensory fibers, so the stimulation amplitude can be lower than similar sensory cases.

As used herein, the term “electrode” refers to a conductor through which electricity enters or leaves an object, substance, or region. For example, an electrode can be implanted on the ventrolateral aspect of the spinal cord. The electrode can be any known shape and/or configuration for spinal implantation. For example, an electrode can be a multi-contact lead with a cylindrical shape. In another example, an electrode can be one or more paddle shaped leads.

As used herein, the term “neurological impairment” can refer to a disorder due to a disease, an injury, a condition, or the like, that can negatively affect one or more motor functions and includes at least one element related to the central nervous system (CNS). Example disorders include stroke, spinal cord injury, and/or another disorder of the CNS.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

As used herein, the term “medical professional” can refer to an individual who provides care to a patient. A medical professional can be, for example, a doctor, a physician's assistant, a student, a nurse, a caregiver, or the like.

### II. Overview

Stroke is the leading cause of serious long-term disability in the United States, with approximately 800,000 people a year having a stroke in the US alone. Strokes often impair motor functions and cause reduced mobility in more than half of stroke survivors aged 65 and older. Spinal cord injury or spinal dysfunction affects over a quarter of a million people in the United States and can significantly negatively affect motor functions-including causing partial or full paralysis. Traditionally, restoring one or more motor functions to a patient after a stroke, spinal cord injury, and/or another disorder of the central nervous system (CNS), can be difficult and, in severe cases, impossible.

A new way to restore one or more motor functions to the patient is via ventral spinal cord stimulation (SCS). One or more stimulating electrodes can be positioned within a ventral space of the patient's spinal column (e.g., arranged at one or more levels of the spinal cord corresponding to different motor functions that are lacking), and in communication with a signal generator, to deliver an electrical stimulation signal to one or more motor fibers in the spinal column. The one or more stimulating electrodes can be positioned within the ventral space proximal to one or more descending spinal tracts that include the one more motor fibers. The electrical signal can be applied in response to a manual input and/or in response to feedback from a sensor (e.g., based on one or more physiological signals of the patient). For instance, the electrical signal can be applied to stimulate motor fiber(s) in a descending tract of the spinal cord of the subject based on the at least one input, the input can change, and the electrical signal and/or the electrode that delivers the electrical signal can correspondingly alter in response to receiving the changed input in a feedback loop. Motor functions that may be at least partially restored can include but are not limited to controlled movement in one or more portions of the lower limbs, controlled movement in one or more portions of the upper limbs, control of one or more organ functions, or the like.

### III. System

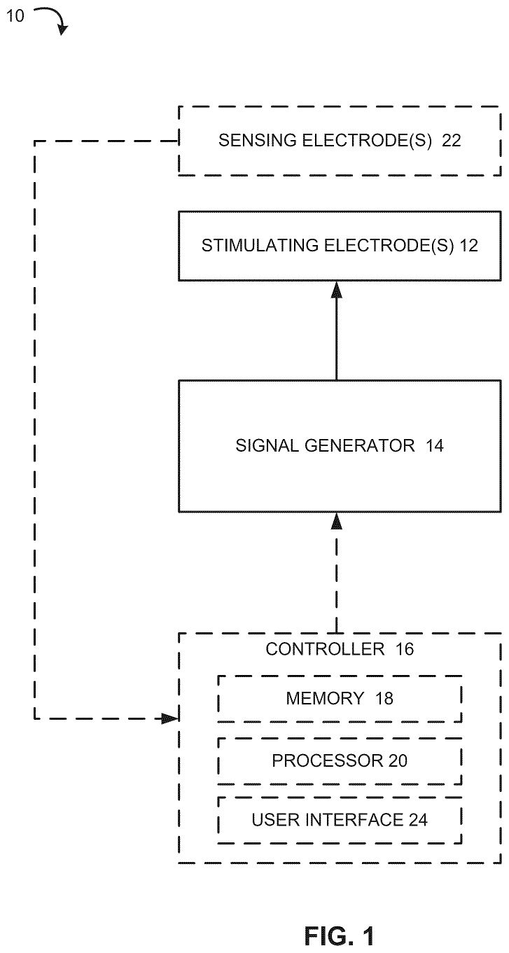

Provided herein is a system 10 (FIG. 1) for ventral Spinal Cord Stimulation (SCS), where one or more stimulating electrodes 12 can be positioned in the ventral space of a spinal cord to provide electrical stimulation. An example of the ventral SCS can be ventrolateral SCS, where one or more stimulating electrodes 12 can be positioned laterally (on either or both sides) of the spinal cord in the ventral space to provide electrical stimulation. Ventral SCS can be used as a treatment to restore one or more motor function(s) that have been lost or impaired in a user suffering from stroke, spinal cord injury (SCI), and/or other disorders of the central nervous system (CNS). While not wishing to be bound by theory, it is postulated that ventral stimulation can directly modulate descending tracts of the spinal cord that can trigger one or more motor functions.

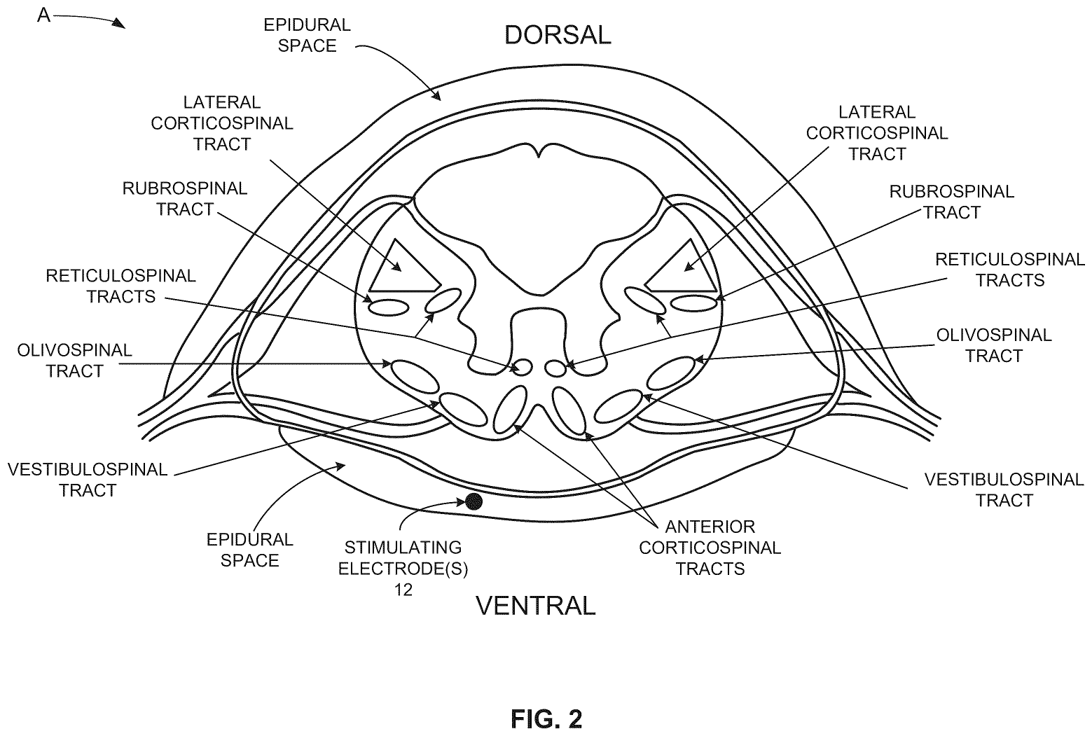

Descending tracts (also referred to as motor tracts) in the spinal cord are the pathways by which motor signals are sent from the brain through the spinal cord to peripheral nerves and communicated to one or more muscles. The descending tracts can be classified by their structural arrangement as lateral tracts and medial tracts and can also be divided functionally into two major groups: pyramidal tracts (which travel from the cerebral cortex through the pyramids of the medulla and include fibers that carry signals to directly innervate motor neurons) and extrapyramidal tracts (generally modulate and indirectly control anterior (ventral) horn cells to modulate motor activity without directly innervating motor neurons). In the spinal cord, the pyramidal tracts can include the lateral corticospinal tract and the anterior corticospinal tract (general positioning of these tracts in the spinal cord is shown in FIG. 2). The extrapyramidal tracts, in the spinal cord, can include the rubrospinal tract, the reticulospinal tracts (lateral and medial), the olivospinal tract, and the vestibulospinal tract (general positioning of these tracts in the spinal cord is shown in FIG. 2). It should be understood that as discussed herein the descending tracts can refer to one or more tracts within the pyramidal and/or extrapyramidal tracts of the spinal cord depending on the motor function(s) to be at least partially restored. Each of the descending tracts can include one or more motor fibers that may be stimulated separately and/or as a group of two or more. Ventral SCS can directly modulate one or more motor fibers within one or more descending tracts of the spinal cord (e.g., the anterior corticospinal tracts, the vestibulospinal tract, or the like). Ventral SCS can be used to at least partially restore motor functions (and/or control motor functions) controlled by voluntary muscles (e.g., consciously controlled skeletal muscles) and/or involuntary muscles (e.g., unconsciously controlled smooth or cardiac muscles) depending on the location(s) and/or configurations of the ventral SCS.

The system 10 can include at least one stimulating electrode (stimulating electrode(s) 12) electrically connected to a signal generator 14 (e.g., wired and/or wirelessly connected). The stimulating electrode(s) 12 can be positioned at one or more locations in a ventral space of the spinal column of a subject (e.g., the ventral epidural space or on the ventral side of the spinal column). The stimulating electrode(s) 12 can be positioned by surgical implantation. It should be understood that the stimulating electrode(s) 12 can be implanted at one or more locations in the ventral space (e.g., any location(s) in the ventral epidural space or on the ventral side of the spinal column) and can be implanted at one or more levels of the spinal column or cord, vertebrae, and/or other identifiable anatomical location depending on the location of the motor function being restored. For example, the stimulating electrode(s) 12 can be implanted at one or more of the cervical levels (C1-C8), the thoracic levels (T1-T12), the lumbar levels (L1-L5), the sacral levels (S1-S5), or the coccygeal segment to affect various motor functions known to be associated with those levels. For instance, if the stimulating electrode(s) 12 are positioned at a thoracic level of the spine the ventral SCS can at least partially restore motor functions in the lower body (e.g., standing, locomotion, joint control, coordination, etc.). In another instance, if the stimulating electrode(s) 12 are positioned at a cervical level of the spine the ventral SCS can at least partially restore motor function in the upper limbs (e.g., reaching, grasping, lifting at least one arm, etc.). The positioning of the stimulating electrode(s) 12 can be determined based on the portion of the body of the patient affected by the stroke, spinal cord injury, and/or the other disorder of the CNS. In some instances, the stimulating electrode(s) 12 can be positioned on one or both lateral sides depending on the impaired motor function (e.g., both lower limbs effected, only one lower limb effected, or the like). As an example, placement the stimulating electrode(s) 12 can be at least partially determined (e.g., by at least one medical professional) based on the American Spinal Injury Association (ASIA) Classification of the ten muscle groups representing motor innervation by the cervical and lumbosacral spinal cord.

The stimulating electrode(s) 12 can be, for example, at least one percutaneously implanted electrode (e.g., cylindrical electrode), at least one implantable paddle electrode, or the like. The stimulating electrode(s) 12 can be at least partially made of and/or (at least substantially) coated with any known biologically safe, conductive material. As an example, the stimulating electrode(s) 12, can be at least partially made of platinum-iridium. The stimulating electrode(s) 12 can include one or more electrical contacts. For example, the stimulating electrode(s) 12 can be a lead having 8 electrical contacts. When the one or more electrical contacts is a plurality of electrical contacts, the electrical signal can be applied via one or more of the plurality of electrical contacts. In some instances, the best/most ideal one or more electrical contacts of the plurality of electrode contacts for the stimulating electrode(s) 12 to deliver the stimulation that restores the muscle function for the patient the best can be determined by one or more processes (e.g., guess and check, verbal feedback, physiological feedback, etc.).

The stimulating electrode(s) 12, can apply at least one electrical signal to at least one motor fiber in a descending tract (e.g., a descending tract within electrical communication access from the position of the stimulating electrode(s)) to at least partially restore at least one motor function of the subject. For example, the motor fibers can be within an anterior descending tract of the spinal cord (e.g., one or more of the anterior corticospinal tracts). The signal generator 14 can generate the electrical signal and communicate the at least one electrical signal to the stimulating electrode(s) 12 to stimulate the at least one motor fiber in the descending tract of the spinal cord. The controller 16 can be in electrical communication (wired and/or wireless with the signal generator 14 and can configure the at least one electrical signal and communicate the configuration to the signal generator.

The signal generator 14 can be at least partially implanted and/or at least partially external and can be connected to the stimulating electrode(s) 12 by a wired connection and/or a wireless connection. For example, the signal generator 14 can be an implanted signal generator (such as an implantable pulse generator) and/or an external signal generator. In some instances, the signal generator 14 can include a rechargeable power source (e.g., a battery). The signal generator 14 can generate one or more electrical signals with one or more parameters, which can be pre-programmed and/or selected by the controller 16. For example, the electrical signal(s) can have a frequency between 0.001 MHz and 1 MHz and a current between 0.001 mA and 50 mA, between 0.001 mA and 20 mA, between 0.001 mA and 10 mA, or preferably between 0.001 mA and 5 mA. While not wishing to be bound by theory, it is believed that there is a lower activation threshold for motor neurons to receive and produce a motor output in at least one muscles compared to sensory fibers (e.g., for restoring and/or enhancing sensation). The electrical signal(s) can be an alternating current or a direct current waveform, a monophasic or biphasic waveform. The electrical signal(s) can be applied continuously or pulsed, and in the case of more than one electrical signal can be applied concurrently, consecutively, and/or with an overlap time. The electrical signal(s) can be generated and applied for a time period ranging from 1 second to all day (e.g., 24 hours) to at least partially restore one or more motor functions of the patient. The configuration and/or application of the electrical signal(s) can be pre-set (e.g., by a medical professional) and/or delivered at the request of the patient (although a maximum safe delivery amount for a period of time might be set) to at least partially restore the motor function of the subject.

The system 10 can include controller 16 electrically connected to the signal generator 14 by a wired and/or wireless connection. The controller 16 can include a non-transitory memory (e.g., memory 18) that can store one or more instructions and a processor 20 that can execute the instructions. The memory 18 and processor 20 can be embodied separately and/or in a combination device such as a microprocessor. The controller 16 can also include a user interface 24 (for bi-directional communication, e.g., a visual display with at least one of touch screen, a cursor, a keyboard, buttons, or the like, an audio output, and/or the like). The user interface 24 can receive an input (e.g., manual input from a user (the subject and/or a medical professional) and/or input from one or more sensors (e.g., time based, biologically based, or the like). The input can include a trigger to begin, end, or alter the electrical stimulation and/or individual electrical signal(s) within the electrical stimulation. The controller 16 can be, for example, external to the patient's body, such as a smart phone, a tablet, a computer, a dedicated external device, or the like. The controller 16 can execute instruction that can include programing one or more parameters of the electrical signal(s) generated by the signal generator 14. The one or more parameters can be pre-loaded in the memory 16, selected based on values and/or ranges in a look up table stored in the memory in response to a circumstance (e.g., based on one or more physiological recordings from one or more sensing electrode(s) 22) and/or based on manual input (e.g., from a user interface 24 associated with the controller 14).

The system can include one or more sensing electrode(s) 22 in electrical communication with at least the controller 16. The one or more sensing electrode(s) 22 can record biological data (e.g., physiological signals) of the subject related to the motor function (e.g., evoked compound action potentials and/or muscle force recordings). For instance, the sensing electrode(s) 22 can be placed near, on, and/or within at least one nerve and or at least one muscle fiber associated with a desired motor function. In some instances the sensing electrode(s) 22 can be another type of sensing element (not shown) such as, but not limited to, an accelerometer, a gyroscope, an inertial measurement unit, a position sensor, or the like that can sense and record one or more parameters associated with the one or more intended motor function (e.g., recording leg movement speed for walking, recording finger, hand, and/or arm positioning, velocity, or the like for reaching and/or grabbing), In other instances, the sensing element can be clock and the system 10 can deliver the stimulation based on at least partially time-based stimulation control (e.g., provide X stimulation at A time, Y stimulation at B time, and/or no stimulation between C and D times (such as nighttime).

The controller 14 can receive the input (e.g., a manual input, an input from the one or more sensing element(s), a recoded physiological signal, a timing control, etc.). Based on the input received, the controller 14 can configure at least one electrical signal to stimulate at least one motor fiber in a descending tract of the spinal cord of the subject. For instance, the at least one electrical signal can be based on the at least one recorded physiological signal. The controller 14 can send the at least one electrical signal configuration to the signal generator to be generated and applied through the stimulating electrode(s) 12.

In one instance, the controller 16 can be a closed loop controller and can receive at least a recording of one or more evoked compound action potentials from the sensing electrode(s) 22, modulate at least one parameter of the electrical signal(s) in response to the one or more evoked compound action potentials from the sensing electrode(s) 22 to improve the restoration of muscle function, and send the modulated at least one parameter to the signal generator 14 to update the electrical signal(s). The sensing electrode(s) 22 may additionally and/or alternatively record electrical signals related to muscle contractions. Additionally or alternatively to the one or more sensing electrode(s) 22, other sensing elements can also be used that can detect one or more other physiological signals, movement parameters, or the like that may indicate a need for a muscle function to be restored and/or that the electrical stimulation should be altered (e.g., the muscle function sending the data may be weak (below a threshold) or too strong (above a threshold), a different muscle function should be activated, unintended muscle(s) are innervated, or the like). In some instances, the presence of one or more sensing electrode(s) 22 and the controller 16 can make the system 10 fully and/or at least partially automated (e.g., manual inputs can override and/or alter configuration based on automated configurations).

FIG. 2 shows a representative sketch of a cross section of the patient's spinal cord with the epidural space shown (may not be drawn to scale or including all anatomical features and is for illustration purposes only). FIG. 2 shows an example placement of stimulating electrode(s) 12 in the ventral epidural space. FIG. 2, element (A) shows an example where stimulating electrode(s) 12 are positioned in one location in the ventral epidural space. And FIG. 2, element B shows an example where at least two stimulating electrode(s) 12 are positioned at two locations in the ventral epidural space. The stimulating electrode(s) 12 can be positioned at one or more locations within the ventral epidural space and/or on the ventral side of the spinal column, dependent on the motor function being at least partially restored. Positioning stimulating electrode(s) 12 within the ventral space can bring the electrodes significantly closer to particular descending tracts (e.g., the anterior corticospinal tracts, the vestibulospinal tract, the olivospinal tract, the medial reticulospinal tracts, or the like) than traditional dorsal positioning for SCS. This can facilitate a direct block rather than an indirect or partial block. In some instances, the stimulating electrode(s) 12 can be placed on one or more portions the spinal column itself.

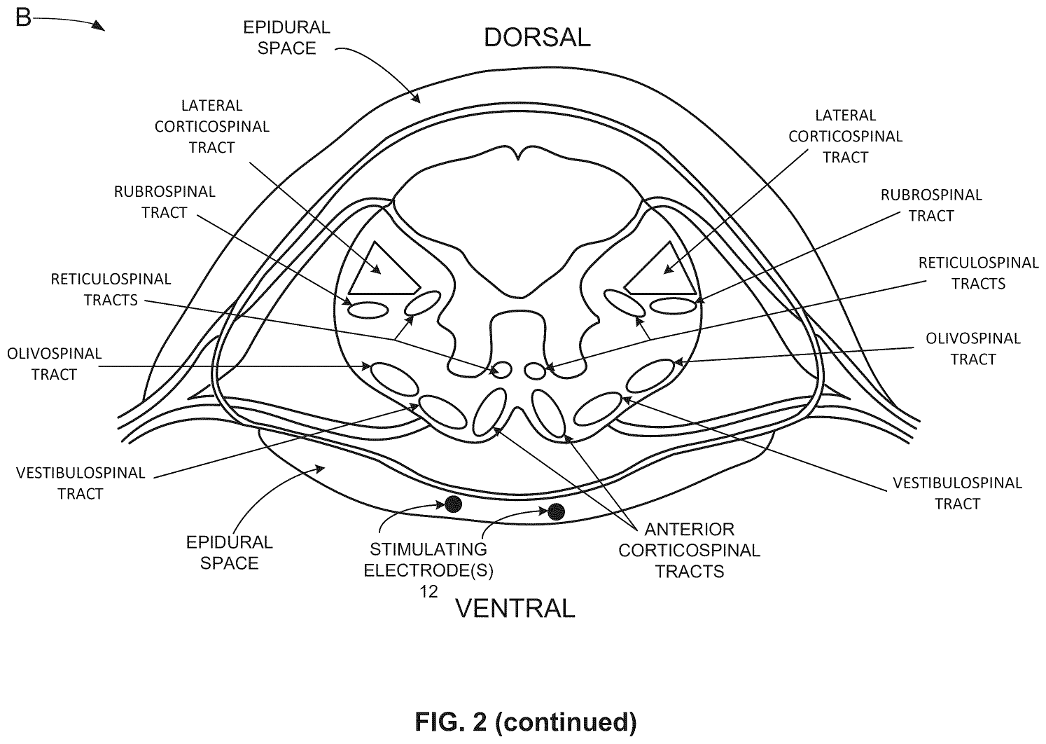

As shown in FIG. 2, element A, the stimulating electrode(s) 12 can be positioned at a more medial portion of the ventral epidural space to be closer, for instance, to the vestibulospinal tract and/or one of the anterior corticospinal tracts. In some instances, a subject only requires restoration of motor function for a specific portion and/or side of the body and the stimulating electrode(s) can be positioned accordingly. The stimulating electrode(s) 12 can be positioned on the right and/or left lateral sides, as shown in FIG. 2, element B, when, for instance, restoration of motor function on both sides of the subject's body is desired. Each of the stimulating electrode(s) 12 can apply the same and/or different electrical signals in a pattern, concurrently, consecutively, and/or at least partially overlapped. As shown and described in more detail with respect to FIG. 3, one or more additional stimulating electrodes may also be positioned in the dorsal epidural space to provide additional electrical stimulation closer to the more dorsal and/or central descending tracts (e.g., the lateral corticospinal tract, the rubrospinal tract, the lateral reticulospinal tracts, etc.).

The following are partial, general lists of which descending tracts can affect which motor functions and/or muscle(s) (please note these are for example purposes only and are not exhaustive). The anterior corticospinal tract (on each side) can, for instance, include motor fibers that are at least partially involved in control of voluntary muscles of the trunk, neck, and shoulders. The lateral corticospinal tract (on each side) can, for instance, include motor fibers that are at least partially involved in control of voluntary muscles of the limbs. It should be noted, while not wishing to be bound my theory, that out of all corticospinal fibers approximately 20% terminate at thoracic levels, 25% at lumbosacral levels and 55% at cervical levels and the level at which the stimulating electrode(s) 12 are positioned can play a part in which muscle(s) are stimulated and/or motor functions are restored. The vestibulospinal tracts include both medial and lateral tracts and are essential for a number of reflex actions of the body (e.g., posture maintenance, balance maintenance, head and/or eye coordination, or the like) by increasing muscle tone in antigravity muscles and/or extensor muscles. The vestibulospinal tracts generally innervate interneurons in the cervical and upper thoracic spine. The olivospinal tract, whose existence may have been disproven, may descend to the lateral funiculus of the spinal cord and modulate activity of spinal anterior grey column motor neurons. The reticulospinal tracts include medial and lateral tracts and are believed to be primarily responsible for locomotion and for postural control as well as involved in controlling sympathetic and parasympathetic outflows and autonomic functions such as heart rate, circulation, and breathing. The rubrospinal tract is involved in controlling involuntary movements (e.g., controlling spasticity, tics, etc.) and is responsible for flexion and extension tone of large group muscles and fine motor control. The rubrospinal tract primarily terminates at the cervical spinal cord.

FIG. 3 shows another system 30 that governs the utilization of ventral SCS in combination with dorsal SCS. The ventral stimulating electrode(s) 12 can be at least one electrode implanted within the ventral epidural space or on the ventral side of the spinal column (as discussed in detail above). The dorsal electrode(s) 32 can be another at least one electrode implanted within a dorsal portion of the spinal column (e.g., the dorsal epidural space) of the subject and can be used in conjunction with the ventral stimulating electrode(s) 12. The system 30 can include at least one signal generator 14 connected to the at least one ventral SCS electrode (stimulating electrode(s) 12) and, optionally the dorsal electrode(s) 32, but can include a separate signal generator 34 for the dorsal electrodes in some instances. The signal generator(s) 14/34 can be connected to the controller 16 by at least one of a wired and/or wireless connection. The controller 16 can include a user interface 24, a non-transitory memory 18 configured to store instructions and processor 20 configured to execute the instructions. The controller 16 (via processor 20) can at least communicate information from the at least one electrode (stimulating electrode(s) 12) to the at least the other electrode (dorsal electrode(s) 32) and/or to each of the at least one electrode and the at least the other electrode. The information can, for instance, include an indication of a timing for delivering the stimulation and/or whether the at least one electrode and/or the other at least one electrode is delivering the stimulation at a given time and/or a pattern for the stimulations to be applied. Additionally, although not shown, one or more sensing elements can provide input to the controller 16 to help it decide which electrode should stimulate at what stimulation parameters.

### IV. Method

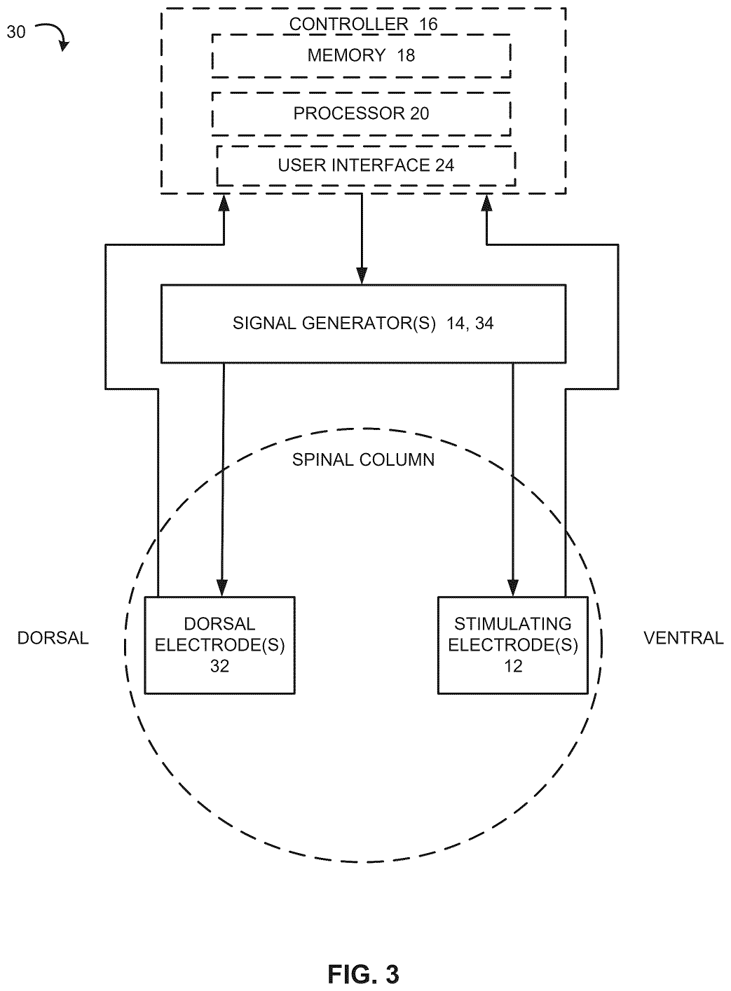

Another aspect of the present disclosure can include methods (FIGS. 4-6) that relate to at least partially restoring motor function with ventral Spinal Cord Stimulation (SCS) that can be implemented with the systems 10 and/or 30 (shown in FIGS. 1 and 3). As an example, the methods 40, 50, and 60 can be executed by controller 16 of systems 10 and 30. In each of the methods 40, 50, and 60, one or more stimulating electrodes 12 of systems 10 and 30 can be positioned within the ventral epidural space of the spinal column (on a level, segment, and/or a side) based on the portion of the body affected by the stroke, spinal cord injury, and/or other disorder of the CNS limiting the motor function of the subject.

The methods 40, 50, and 60 are illustrated as process flow diagrams with flowchart illustrations that can be implemented by one or more components of the systems 10 and/or 30. For purposes of simplicity, the methods 40, 50, and 60 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 40, 50, and 60.

FIG. 4 shows method 40 for using ventral SCS to at least partially restore a subject's motor function that was impaired and/or lost due to stroke, spinal cord injury, or another disorder of the central nervous system (CNS). At 42, at least one electrode (e.g., stimulating electrode(s) 12) can be implanted within a ventral space (e.g., within the ventral epidural space and/or on the ventral side of the spinal column) of a spinal column of the subject. The at least one electrode can be implanted by any surgical means. The at least one electrode can be positioned proximal to one or more descending tracts of the spinal cord that can trigger one or more motor functions (e.g., an anterior corticospinal tract, a vestibulospinal tract, etc.). If the at least one electrode is more than one, then each of the electrodes can be positioned proximal to at least one of the one or more descending tracts at the same or different levels of the spine and/or different medial/lateral positions depending on the motor function(s) being at least partially restored. For example, an electrode can be positioned in the ventrolateral epidural space as shown in FIG. 2. The at least one electrode can be implanted at any vertebrae or segment at any level of the spine (e.g., thoracic, lumber, sacral, cervical, coccygeal, etc.) dependent on the motor function to be at least partially restored. For example, the at least one electrode can be implanted on at least one thoracic level of the spine to at least partially restore motor function in at least a portion of the back and/or lower limbs. In another example, the at least one electrode (stimulating electrode(s) 12) can be implanted on at least one cervical level of the spine to at least partially restore motor function of at least a portion of the upper limbs.

At 44, an electrical signal (at least one electrical signal) can be generated by the signal generator (e.g., signal generator 14) to stimulate at least one motor fiber to at least partially restore muscle function. The electrical signal(s) can be generated with one or more parameters (e.g., based on a user input, a preprogrammed time for stimulation, an input from one or more sensing elements (e.g., evoked action potentials, muscle contraction information, position information, velocity information, or the like). For instance, the stimulation can be started or ended based on a user input. The one or more parameters of the electrical signal(s) (e.g., amplitude, power, frequency, continuous, pulsing, timing, etc.) can be pre-programmed and/or selected by a controller (e.g., controller 16). In some instances, one or more parameters of the electrical signal(s) can be modified during application based on the user input, input from one or more sensing elements, etc. The electrical signal can be an alternating current or a direct current waveform, a monophasic or biphasic waveform.

At 46, the electrical signal can be applied via that at least one electrode to stimulate the at least one motor fiber to at least partially restore muscle function. The electrical signal can be applied continuously or pulsed. The electrical signal can be generated and applied for a time period ranging from 1 second to all day (e.g., 24 hours) to at least partially restore at least one motor function of the subject. For example, the electrical signal can be applied to stimulate the at least one motor fiber to restore the muscle function continuously for a time period. The time period can be started and/or stopped manually by the patient and/or can be a predetermined time period programmed into the system (e.g., system 10).

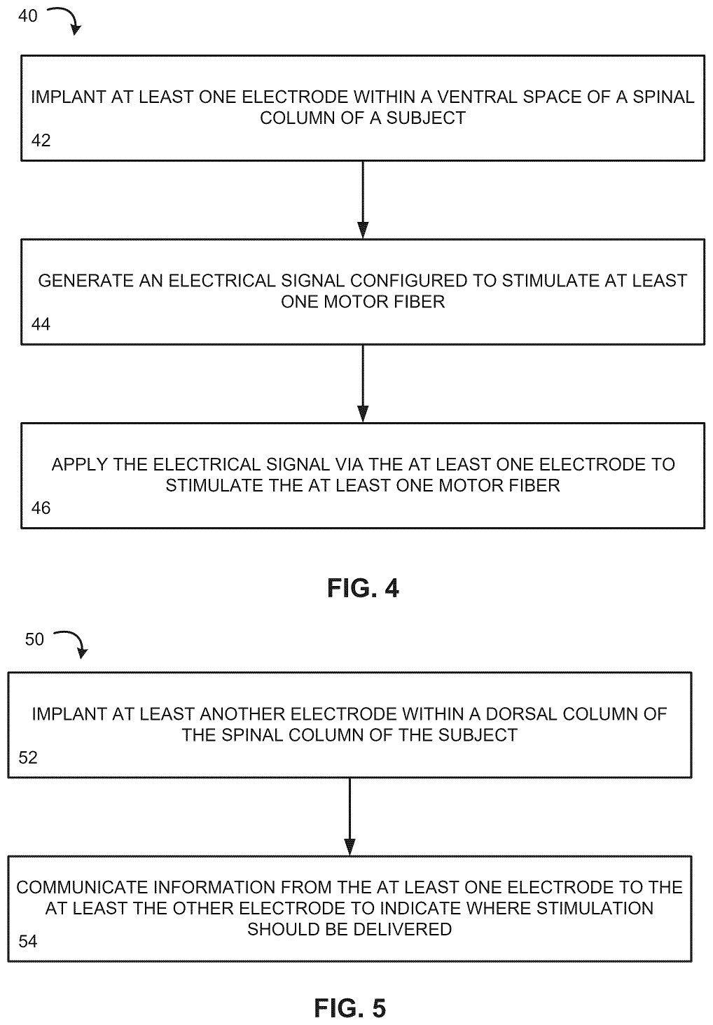

Method 50 shown in FIG. 5 can be used with method 40 in order to apply both ventral SCS and dorsal SCS to a subject to restore a patient's motor function. At 52, at least another electrode (e.g., dorsal electrode(s) 32) can be implanted within a dorsal portion of the spinal column of the subject (e.g., within the dorsal epidural space). The at least the other electrode (e.g., dorsal electrode(s)) can be implanted at the same time, prior to, and/or after the at least one electrode (e.g., ventral stimulating electrode(s)). The at least one (ventral) electrode and the at least the other (dorsal) electrode can be positioned at a same level and/or segment of the spine and/or at different levels and/or segments of the spine. Both the at least one electrode (e.g., ventral stimulating electrode(s)) and the at least the other electrode (e.g., dorsal electrode(s)) can be electrically connected (wired and/or wirelessly) to a system (e.g., at least one controller, including a processor, and signal generator). At 54, information can be communicated, by the system, from the at least one electrode (e.g., ventral stimulating electrode(s)) to the at least the other electrode (e.g., dorsal electrode(s)), and vice versa, to indicate where the stimulation should be delivered and/or one or more parameters for configuring the simulation. The information can also include, for instance, include an indication of a timing for delivering the stimulation and/or whether the at least one electrode and/or the other at least one electrode is delivering the stimulation at a given time. For example, stimulation can be alternated between dorsal and ventral electrodes, changed from one dorsal electrode to another or from one ventral electrode to another, and/or at least partially simultaneously applied through both dorsal and ventral electrode(s).

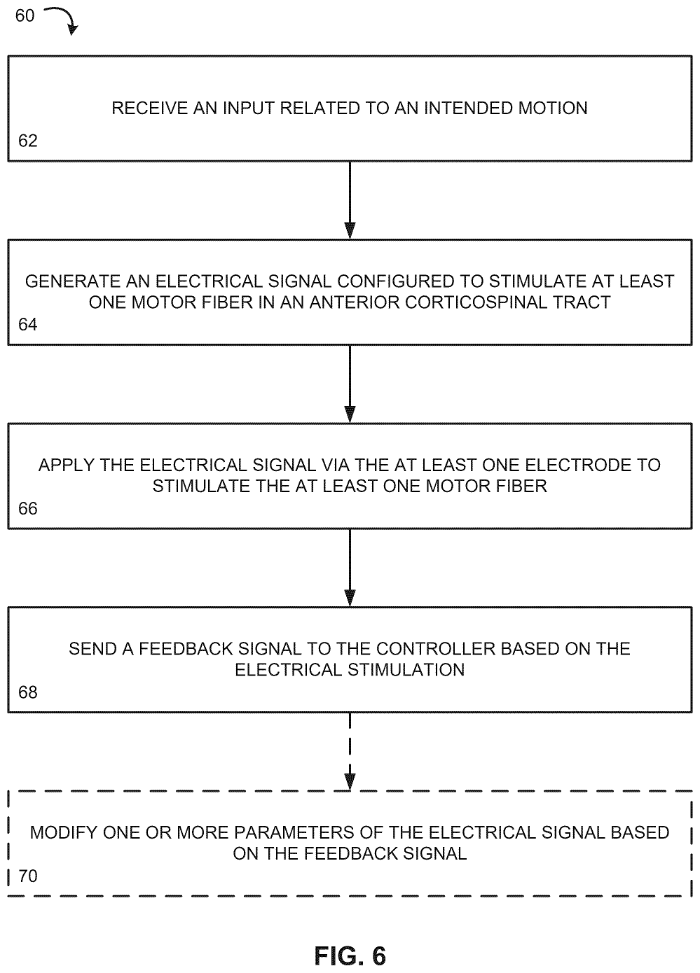

Method 60 of FIG. 6 shows a method for controlling ventral SCS with feedback using a system 10 including a controller having a non-transitory memory and a processor (e.g., controller 16, memory 18, and processor 20, as well as a user interface 24 and/or sensing element(s) (e.g., sensing electrode(s) 22)). At 62, an input can be received (e.g., a user input, an input from one or more sensing elements, etc.) related to an intended motion of a subject. If the input is from one or more sensing elements, the input can be based on at least one recorded physical signal (e.g., an evoked action potential, a recording of muscle force, etc.), one or more movement parameters, or the like. For example, the sensing element(s) can be at least one of an electrode, a gyroscope, an accelerometer, a position sensor, or the like. The input can be a manual input related to the at least one motor function from the user that the user inputs through a user interface (e.g., a touch screen, a keyboard and/or mouse, button(s), a microphone, or the like). In other instances, the input can be time based and can be provided by a clock in communication with the controller. In some instances, the input can be part of an automatic or semi-automatic control loop (e.g., based on time of day, common activities, feedback from a sensing element not matching an intended motor function, or the like).

At 64, an electrical signal (at least one electrical signal) can be generated by a signal generator that is configured (by the controller) to stimulate at least one motor fiber in an anterior corticospinal tract (or other descending tract). The configuration of the electrical signal can be transmitted from the controller to the signal generator based on the input and/or the motor function intended to be at least partially restored. At 66, the electrical signal can be applied by the at least one electrode (at least one of the stimulating electrode(s) 12) to stimulate the at least one motor fiber of the anterioro corticospinal tract (or other descending tract). In other words, the at least one electrical signal is transmitted from the signal generator to at least one stimulating electrode within a ventral space of a spinal column of a subject affected by stroke and/or spinal cord injury for application to the at least one motor fiber to at least partially restore the at least one motor function of the subject. At 68, a feedback signal (e.g., from one or more sensing element(s) 22) can be sent to the controller based on the electrical stimulation being applied. Optionally, at 70, the controller (e.g., controller 16) can modify one or more parameters of the electrical signal based on the feedback that is received and send the modified configuration to the signal generator, which can then output the modified electrical signal to the at least one electrode for application. The one or more parameters modified can include, but are not limited to, the frequency, the amplitude, the timing, and/or the location of application of the electrical signal.

From the above description, those skilled in the art will perceive improvements, changes and modifications. Such improvements, changes and modifications are within the skill of one in the art and are intended to be covered by the appended claims. All patents, patent applications, and publications cited herein are incorporated by reference in their entirety.

What is claimed is:

1. A method comprising:
receiving, by a controller comprising a processor, an input based on at least one motor function of a subject;
configuring, by the controller, at least one electrical signal designed to stimulate at least one motor fiber in a descending tract of the spinal column and/or spinal cord; and
sending, by the controller, a configuration of the at least one electrical signal to a signal generator,
wherein the at least one electrical signal is transmitted from the signal generator to at least one stimulating electrode within a ventral space of a spinal column of a subject affected by stroke and/or spinal cord injury for application to the at least one motor fiber to at least partially restore the at least one motor function of the subject.

2. The method of claim 1, wherein the receiving the input further comprises receiving the input from at least one sensing element, wherein the at least one sensing element records at least one physiological signal related to the at least one motor function of the subject.

3. The method of claim 2, wherein the at least one sensing element recording comprise an evoked compound action potential and/or a recording of muscle force.

4. The method of claim 1, wherein the receiving the input further comprises receiving a manual input related to the at least one motor function of the subject.

5. The method of claim 1, wherein the input is part of an automatic control loop configured to provide a time based motor-function.

6. The method of claim 1, wherein the at least one stimulating electrode is positioned within the ventral space of the spinal column proximal to the descending tract.

7. The method of claim 6, wherein the descending tract is an anterior descending tract.

8. The method of claim 7, wherein the anterior descending tract is an anterior corticospinal tract.

9. The method of claim 1, wherein the at least one stimulating electrode is positioned within the ventral space of the spinal column at a location determined based on the portion of the body affected by the stroke and/or the spinal cord injury limiting the motor function of the subject.

10. A system comprising:
one or more sensing elements configured to record one or more physiological signals from a subject;
a signal generator configured to produce at least one electrical signal;
one or more stimulating electrodes configured to be positioned within a ventral space of a spinal column of the subject and in electrical communication with the signal generator;
a controller connected to the one or more sensing elements and to the signal generator and comprising:
a non-transitory memory configured to store instructions, and
a processor configured to execute the instructions to at least:
receive a signal comprising at least one recorded physiological signal from at least one of the one or more sensing elements;
configure at least one electrical signal to stimulate at least one motor fiber in a descending tract of the spinal cord of the subject based on the at least one recorded physiological signal; and
send the at least one electrical signal to the signal generator to be applied through at least one of the one or more stimulating electrodes,
wherein the at least one of the one or more stimulating electrodes are configured to apply the at least one electrical signal to the at least one motor fiber to at least partially restore a motor function of the subject.

11. The system of claim 10, further comprising a user interface connected to the controller and configured to receive an input comprising a trigger to begin or to end electrical stimulation.

12. The system of claim 10, further comprising one or more other stimulating electrodes configured to be positioned in a dorsal space of the spinal column and used in conjunction with the one or more stimulating electrodes positioned within the ventral space of the spinal column.

13. The system of claim 12, wherein the one or more other stimulating electrodes are activated to apply at least one other electrical signal in a pattern with the one or more stimulating electrodes.

14. The system of claim 10, wherein the one or more stimulating electrodes are configured to be positioned at one or more levels of the spinal column and/or spinal cord.

15. The system of claim 14, wherein the one or more stimulating electrodes are configured to be positioned on at least one cervical level, at least one thoracic level, and/or at least one lumbar level of the spinal column and/or spinal cord.

16. The system of claim 14, wherein the one or more stimulating electrodes are configured to be positioned within the ventral portion of the spinal column based on a portion of the body affected by a stroke and/or spinal cord injury limiting the motor function of the subject.

17. The system of claim 10, wherein the subject is affected by stroke and/or spinal cord injury.

18. The system of claim 10, wherein the one or more sensing elements comprise at least one electrode configured to be placed proximal to, on, and/or within at least one nerve and/or at least one muscle fiber.

19. The system of claim 10, wherein the one or more physiological signals comprise evoked compound action potentials and/or muscle force recordings.

20. The system of claim 10, wherein the one or more stimulating electrodes are configured to be positioned at a thoracic level to effect motor function in a lower body of the subject and wherein the one or more stimulating electrodes are configured to be positioned at a cervical level to effect motor function in one or more upper limbs of the subject.
